(19) United States
(12) Patent Application Publication
(10) Pub. No.: US 2012/0272989 A1
(43) Pub. Date: Nov. 1, 2012

(54) Hair Removal Method and Kit

(76) Inventor: Charles Robert SMITH, Henley on Thames (GB)

(21) Appl. No.: 13/458,190

(22) Filed: Apr. 27, 2012

Related U.S. Application Data
(60) Provisional application No. 61/479,876, filed on Apr. 28, 2011.

(51) Int. Cl. A61K 8/92; A61Q 9/04

(52) U.S. Cl. 132/202; 8/161; 132/320

(57) ABSTRACT

A method of removing hair from skin is provided comprising the steps of:

(a) applying a protective composition to an area of skin on which unwanted hair is growing, wherein the protective composition comprises at least 75% petrolatum by weight of the protective composition, the protective composition having a cone penetration of 160 to 210;

(b) applying a depilatory composition to the area of skin to which the protective composition has been applied, the depilatory composition comprising a keratin reducing agent.

### CROSS REFERENCE TO RELATED APPLICATION

This application claims the benefit of U.S. Provisional Application No. 61/479,876, filed 28 Apr. 2011.

### FIELD OF THE INVENTION

The present invention relates to a method and kit for depilation.

### BACKGROUND OF THE INVENTION

Depilatory compositions are cosmetic hair removal formulations. They comprise keratin reducing agents, which attack the disulphide bonds in hair to weaken it, such that subsequent gentle scraping and/or wiping completes severance of the hair from the skin and effects hair removal. Commercially, the most common keratin reducing agents are thioglycolates, which are typically formulated at high pH. An unwanted side effect of chemical depilation is that the depilatory composition comes into contact with and must have a relatively long residence time on skin to achieve effective hair removal and this long residence time combined with the alkaline conditions needed for effective hair removal may give rise to skin irritation.

The above problem has been recognized in the art. Reference is made to U.S. Pat. No. 4,401,663 and U.S. Pat. No. 4,424,205 the disclosures of which are similar to one another. These documents teach to use certain compounds as topical analgesics and an example given of a situation in which it is suggested to use such materials is to reduce depilatory irritation. The carrier formulas disclosed to be suitable for formulation with the analgesics include lotions and creams.

Reference is also made to US 2004/0219118, which discloses treatment with a “lipophilic” material before application of a thioglycolate-based reactive depilatory composition. Lipophilic materials exemplified in this patent application are oils, such as mineral oil. As shown hereinbelow, the present applicants have tested a range of lipophilic materials to determine their ability to prevent thioglycolate penetration and, thereby, their ability to reduce or prevent skin irritation Applicants have surprisingly found that oils, such as mineral oil, have no or a low ability to prevent thioglycolate penetration to the skin. There thus exists a need to develop a pre-treatment composition which better reduces skin irritation.

### SUMMARY OF THE INVENTION

According to a first aspect of the invention, a method of removing hair from skin is provided, comprising the steps of:
(a) applying a protective composition to an area of skin on which unwanted hair is growing, wherein the protective composition comprises at least 75% petrolatum by weight of the protective composition, the protective composition having a cone penetration of 160 to 210;
(b) applying a depilatory composition to the area of skin to which the protective composition has been applied, the depilatory composition comprising a keratin reducing agent.

According to a second aspect of the invention, a depilatory kit is provided, comprising:
(a) a protective composition, the protective composition comprising at least 75% petrolatum by weight of the protective composition, the protective composition having a cone penetration of 160 to 210;
(b) a depilatory composition comprising an effective amount of a keratin reducing agent.

### BRIEF DESCRIPTION OF THE DRAWINGS

FIG. 1. is a schematic view of a Franz Cell apparatus.

### DETAILED DESCRIPTION OF THE INVENTION

The protective composition used in the method and comprised within the kit according to the invention comprises at least 75% petrolatum by weight of the protective composition, the protective composition having a cone penetration of 160 to 210. Applicants have found, as demonstrated by the Franz Cell data presented herein, that a composition having the defined cone penetration, which is a measure of hardness and comprising at least 75% petrolatum, inhibits, penetration by thioglycolic acid versus prior art compositions, such as the oils disclosed in US 2004/0219118 discussed above, to a surprising degree. A reduction of thioglycolic acid penetration of 45% or more according to the Franz Cell method may be shown to correlate to a significant and user-noticeable reduction in irritation. At the same time, applicants have established that the use of the present protective composition still permits hair weakening and destruction by the keratin reducing agent, so that reduced skin irritation can be achieved while removing hair. A cone penetration of less than 160 defines a protective composition which is too difficult to spread and will generally reduce hair removal efficacy and a cone penetration of above 210 defines a composition which is too thin to generate a coherent film that provides an effective barrier, resulting in an increase in irritation.

As used herein, the term “cone penetration” refers to cone penetration as measured by the method of ASTM Standard D937, 2007, “Standard Test Method for Cone Penetration of Petrolatum” ASTM International, West Conshohocken, Pa., 2003, DOI: 10.1520/D0937-07, www.astm.org. This method was devised to measure petrolatum alone, but may be used to measure the cone penetration of the present compositions, given the high level of petrolatum comprised within them. For completeness, commercially available petrolatum has quoted cone penetration values in the range from 70 to 350. Cone penetration in the D937 ASTM standard state is measured as the number of 1/10th millimeters, so that, by way of example, a “200” value of cone penetration equates to a 20 mm penetration.

Reference is also made to the following standard for the equipment needed (such as the cone and equipment specifications): ASTM Standard D217, 2010, “Standard Test Methods for Cone Penetration of Lubricating Grease” ASTM International, West Conshohocken, Pa., 2003, DOI: 10.1520/D0217-10, www.astm.org.

Advantageously, the protective composition comprises at least 80%, preferably at least 90% and more preferably at least 95% petrolatum by weight of the protective composition. While the present compositions may consist entirely of petrolatum (that is, they may be 100% petrolatum), it is preferred that they comprise at least a small proportion of cosmetic excipients, such as perfumes and dyes to make them more pleasant to use.

The protective composition may comprise skin active agents such as, but not limited to oil soluble vitamins, such as vitamin E derivatives, including vitamin E acetate and tocopherol nicotinate; oil-soluble vitamin A derivatives, such as retinyl palmitate; lanolin; ceramides; sterols and sterol esters; salicylic acid; camphor; eucalyptol; essential oils and mixtures thereof.

The protective composition used in the method and comprised within the kit according to the invention may comprise from 1% to 25%, preferably from 1.5% to 15%, more preferably from 3% to 10% of powder to provide a tactile and/or an optical signal to the user allowing them easily to establish to which areas of skin the protective composition has been applied and which not.

In order to have a user-perceptible tactile effect on skin, the powder comprised within the protective composition may advantageously have an average particle size from 0.5 μm to 200 μm, preferably from 1 μm to 130 μm, more preferably from 5 μm to 30 μm.

In order to have a user-perceptible visual effect on skin, the powder comprised within the protective composition may advantageously have an average particle size from 0.05 μm to 200 μm, preferably from 0.1 μm to 130 μm, more preferably from 0.2 μm to 60 μm.

The average particle size is measured using the laser diffraction method defined in ISO13320.

If it is desired to create a user-perceptible visual effect on skin, then, it may be advantageous to use a dye instead of or as well as the powder according to the invention. Any dye which is compatible with the protective composition may be employed and preferably the dye is soluble in wax.

Advantageously, at least some of the particles are coloured to contrast with the skin colour and render them visible to the naked eye. Highly advantageously, they are provided with a colour, such as a green or blue, which is not a human skin colour and may provide a strong contrast to the colour of skin. It is not essential that they be coloured, however, because the particles may provide a tactile signal to the user, whatever colour they are.

The particles may be comprised of any suitable material that may provide a tactile or visible signal to the user and is not otherwise unsuitable, for example because it is an irritant. Suitable materials for the particles may include metals; inorganic materials, such as metal salts and metal oxides, silica, mica and talc; organic materials, such as natural and synthetic polymers.

Exemplary materials from which the particles may be comprised include spherical silica, hydrated silica, silicone treated silica beads, mica, talc, iron oxide, titanium dioxide, zinc oxide, aluminium lake pigments, nylon 12 and nylon 6, polyethylene, starch (such as aluminium starch octenyl succinate), methylsilsequioxane resin, polymethylmethacrylates, crosslinked polydimethylsiloxanes, polyamide, polystyerene, ethylene methacrylate, PTFE, cellulose triacetate and mixtures thereof.

The protective composition used in the method and comprised within the kit according to the invention may additionally comprise a sensate. Sensate materials may provide a sensible signal, such as a cooling or heating sensation, to the user through the skin indicating the presence of the protective composition according to the invention. Such a signal may reinforce the tactile and/or visible signal provided by the powder. Non-limiting examples of sensates include menthol, methone glycerin acetal, methyl diisopropyl propionamide, menthyl lactate, menthoxy-1,2-propanediol, ethyl carboxamide, trimethyl butanamide, menthyl lactate, butanedioic acid monomenthyl ester, vanillyl butyl ether, 1-isopulegol, cineole, methylsalicylate, eucalyptus oil, carvone, rosemary oil, ginger oil, clove oil, ethyl menthane, carboxamide, camphor, eucolytol, peppermint oil, and combinations thereof. If present, the protective composition may comprise from 0.01% to 2.0%, preferably from 0.01% to 1.0% of sensate, by weight of the protective composition.

Any depilatory composition comprising a suitable keratin reducing agent may be used in the present method and included in the present kit. Non-limiting examples of suitable keratin reducing agents include: sulphide salts such as Li₂S, Na₂S, K₂S, MgS, CaS, SrS or BaS, hydrogen sulphide salts such as NaSH or KSH; thioglycol; thioglycerol; thioglycolamide; thioglycolhydrazide; thioglycolic acid; thioglycolate salts (such as potassium thioglycolate, calcium thioglycolate, ammonium thioglycolate, diammonium dithioglycolate, glyceryl monothioglycolate, or monoethanolamine thioglycolate); thiosalicylic acid; thiomalic acid; ammonium thiolactate; monoethanolamine thiolactate; dithioerythritol; 2-mercaptopropionic acid; 1,3-dithiopropanol; glutathione; dithiothreitol; cysteine; homocysteine; N-acetyl-L-cysteine and cysteamine. Advantageously, the keratin reducing agent is comprised within the depilatory composition in an amount from 0.3% to 20%, preferably from 0.8% to 15%, more preferably from 1% to 10% by weight of the depilatory composition.

Advantageously, the depilatory composition may comprise at least one thioglycolate salt or thioglycollic acid acting as a hair removal agent when the depilatory composition is applied to unwanted hair. Preferably, the depilatory composition comprises sodium, potassium, magnesium, calcium, beryllium, strontium, zinc, monoethanolamine, ammonium, tetralkylammonium, imidazolium, pyridinium, phosphonium or glyceryl thioglycolate salts, or mixtures thereof, which may include dianion forms of thioglycolate. More preferably, the depilatory composition comprises at least one of sodium, potassium, magnesium or calcium thioglycolate, or mixtures thereof. Even more preferably the depilatory composition comprises potassium or calcium thioglycolate, or mixtures thereof.

The pH of the depilatory composition may advantageously be in the range of from 6 to 13.8, preferably from greater than 7 to 13, more preferably from 9 to 12.9, even more preferably from 10 to 12.8, even more preferably still from 12 to 12.75 and yet more preferably from 12.3 to 12.6 to improve the efficacy of the active ingredient. The depilatory composition may, in a preferred embodiment, comprise at least one base to control the pH. Preferably, the depilatory composition comprises potassium hydroxide; sodium hydroxide; lithium hydroxide; calcium hydroxide; barium hydroxide; caesium hydroxide; sodium hydroxide; ammonium hydroxide; strontium hydroxide; rubidium hydroxide; magnesium hydroxide; zinc hydroxide; sodium carbonate; pyridine; ammonia; alkanolamides (including monoethanolamine, diethanolamine, triethanolamine), phosphates (including tetrasodium phosphate), arginine or mixtures thereof. More preferably, the depilatory composition comprises at least one buffering base, even more preferably the depilatory composition comprises calcium hydroxide, magnesium hydroxide; barium hydroxide; strontium hydroxide; zinc hydroxide; arginine or mixtures thereof. Still more preferably the depilatory composition comprises calcium hydroxide; magnesium hydroxide, zinc hydroxide, sodium hydroxide, potassium hydroxide or mixtures thereof. Even more preferably still, the depilatory composition comprises calcium hydroxide, sodium hydroxide or mixtures thereof.

In an advantageous embodiment, the base is present at a concentration of from 0.1% to 10.0%, more preferably from 0.5% to 8.0% and even more preferably from 1.0% to 5.0%, by weight of the depilatory composition.

The concentration of water in the depilatory composition is preferably at least 40%, more preferably from 50% to 98%, even more preferably from 60% to 95% and even more preferably still from 70% to 90%, by weight of the depilatory composition.

The depilatory composition may optionally comprise a thickening agent. A representative but not exhaustive list can be found in “The Encyclopaedia of Polymers and Thickeners for Cosmetics” compiled and edited by Robert Y. Lochhead, PhD and William R. Fron, Department of Polymer Science, University of Southern Mississippi. Exemplary classes of thickening agents include gums, carbomers, polymers and copolymers of acrylic acid, associated thickeners, layered silicates/clays and natural polymers (including polysaccharides). One or more thickening agents may be included in the aqueous depilatory composition. The thickening agent may be present at a level of from about 0.01% to about 20%, preferably from about 0.1% to about 10% by weight of the depilatory composition.

The depilatory composition may also include other skin care ingredients such as conditioning agents selected from the group consisting of humectants, moisturizers, or skin conditioners (including mineral oil; almond oil; chamomile oil; jojoba oil; avocado oil; shea butter, niacinamide and glycerine); skin rejuvenation compositions (for example targeted for fine lines, wrinkles and uneven skin tone, including retinoids), cosmetic compositions; anti-inflammatory agents (including corticosteroids); anti-oxidants (including flavonoids) radical scavengers; sunscreen agents; skin cooling or warming agents and the like. The depilatory composition may comprise one or more skin care ingredients present in an amount of from about 0.001% to about 10%, more preferably from about 0.01% to about 7%, and even more preferably from about 0.025% to about 5%, by weight of the depilatory composition.

An accelerant may be employed in the depilatory composition. This optional component accelerates the rate of depilatory action of the depilatory agent. Suitable accelerants include, but are not limited to, urea; thiourea; dimethyl isosorbide; arginine salts; ethoxydiglycol; propylene glycol and methylpropyldiol. The accelerant may be present in a concentration range of from 0.5% to 10%, more preferably from 2% to 8% and even more preferably from 2% to 5% by weight of the depilatory composition.

The depilatory composition may further comprise components known, conventionally used, or otherwise effective for use in cosmetic compositions, such as dyes; pigments (including ultra marines and talc); anionic, cationic, non-ionic and/or amphoteric or zwitterionic surfactants, polymers (including hydrophobically modified polymers); dispersing agents; solvents; lubricants; fragrances; preservatives; chelants, proteins and derivatives thereof, plant materials (e.g. aloe, chamomile and henna extracts); silicones (volatile or non-volatile, modified or non-modified); film-forming agents; film forming promoters and mixtures thereof.

The depilatory composition may be formulated in any common delivery form, such as a cream or lotion. Alternatively, it may be delivered on a substrate, such as a thin film of depilatory composition coated onto the substrate. The substrate may be configured in any suitable form, such as a strip, mask or patch.

In addition to the protective composition and the depilatory composition, the kit according to the second aspect of the invention may comprise one or more of:
(a) A make-up removal composition and/or a make-up removal wipe;
(b) Means for removal of the protective composition and the depilatory composition following use, which means may comprise one or more of a tool, such as a scraper or a spatula; or a wipe;
(c) A post-treatment composition skin care composition to be applied to the area of skin from which hair has been removed. Such a post-treatment skin care composition may comprise ingredients to promote skin conditioning; moisturizers, skin rejuvenation compositions (targeted for fine lines, wrinkles and uneven skin tone, for example), cosmetic compositions (e.g., foundation, rouge), sunscreens and the like. The post-treatment skin care composition may be leave-on or a rinse-off composition.
(d) Instructions regarding how to use the various elements of the kit, which instructions may comprise one or more elements of the method as defined herein.

Prior to applying the method or using the kit according to the present invention, a user should advantageously remove all make-up from the skin, to ensure good adherence and effective application of both the protective composition and the depilatory composition.

The method according to the first aspect of the invention comprises the step of applying the above-defined protective composition to an area of skin on which unwanted hair is growing. The area of skin may be located on any part of the human body.

Advantageously, the protective composition is not just applied to the area to be depilated, but also to an immediately juxtaposing area thereabout (that is, the protective composition is applied to an area of skin which is greater than just the area which is to be depilated).

Advantageously, the user will apply from 0.3-2 mg of protective composition per square centimetre of skin, preferably from 0.4-1 mg/cm², more preferably from 0.4 to 0.7 mg/cm².

Following application, the protective composition is advantageously massaged into the skin. Preferably, massaging is effected for at least 10 seconds, and, more preferably, massaging is effected as a circular motion. Without wishing to be bound by theory, it is believed that the protective composition may trap hair within it thereby shielding it from the to-be-applied depilatory composition; massaging may help to release the hairs from the skin and ensure improved access thereto by the depilatory composition.

The method according to the first aspect of the invention comprises the subsequent step of applying the above-defined depilatory composition to an area of skin on which unwanted hair is growing and to which protective composition has already been applied. Advantageously, the user will apply a layer of depilatory composition which is from 0.1 mm to 5 mm, preferably from 0.3 to 3 mm, more preferably from 0.5 to 2 mm in thickness.

Subsequently, according to the method of the first aspect of the invention, the depilatory composition is advantageously left in place for at least 1 minute, preferably from 1 to 10 minutes, more preferably from 3 to 10 minutes, depending on the thickness of the hair and the hair removal efficacy of the depilatory composition (which, in turn, is dependent upon the concentration of keratin reducing agent in the depilatory composition).

Subsequently, according to the method of the first aspect of the invention, the protective composition and the depilatory composition are advantageously removed. This may be achieved using one or more of a cotton wool ball, pad or wand, a tissue, a cloth, or a tool, such as a spatula or a scraper. Advantageously, the skin from which hair has been removed is then rinsed with water.

In an advantageous subsequent step, a post-treatment skin care composition may be applied to the area of skin from which hair has been removed. Such a post-treatment skin care composition may comprise ingredients to promote skin conditioning; moisturizers, skin rejuvenation compositions (targeted for fine lines, wrinkles and uneven skin tone, for example), cosmetic compositions (e.g., foundation, rouge), sunscreens and the like. The post-treatment skin care composition may be leave-on or a rinse-off composition.

### Franz Cell Method

### Principle and Scope:

This method is applicable for using Franz cell apparatus for the in-vitro assessment of penetration of thioglycolic acid (TGA) and its salts through a skin mimic after the application of a depilatory composition following pre-treatment with a protective composition.

Penetrated TGA is quantified using Reverse Phase High Performance (or Pressure) Liquid Chromatography (RP-HPLC) with external standard quantitation at 240 nm.

### Method

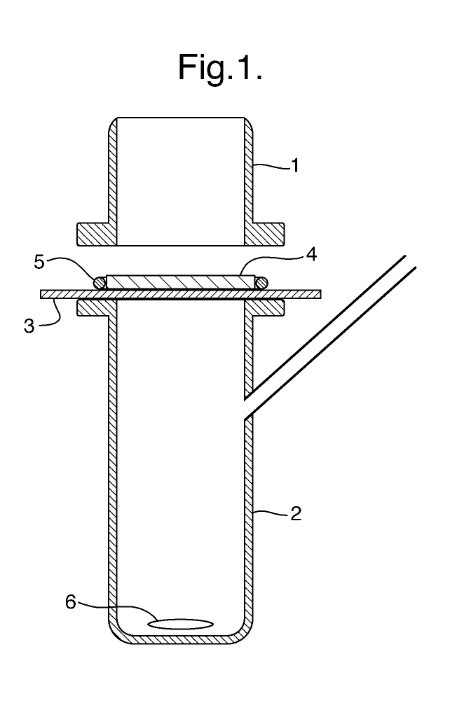

Reference is made to FIG. 1 and to the reference numerals therein:

1. Prepare the Vitro-Skin (IMS Vitro-Skin®, Catalogue number: P&G1013, made by IMS Inc., Portland, Me., USA) samples by cutting 8×6.2 cm segments and placing them textured side up on the racks into a hydration chamber (manufactured & sold by IMS) containing a 14.7% glycerol solution. The hydration chamber should be sealed and the vitro-skin left to hydrate at room temperature and a humidity of 80.4%±3.5% for 24 hours.

2. Prepare the receptor solution for the Franz-cell by mixing 1.90 ml formic acid (98% wt+ Fluka, by Sigma Aldrich, or equivalent), 30 ml acetonitrile (RP-HPLC grade) and 968.1 ml water (RP-HPLC grade). Set up the static Franz cell (Permegear or equivalent, 15 mm diameter unjacketed cell with a 12 ml receptor volume) by clamping it in place over suitable stirrer plates (not shown) and add a small stirrer bar (6) to each cell, fill the receptor cell (2) to the brim with the required amount of receptor solution.

3. Once hydrated, remove a sheet of vitro-skin from the hydration chamber and lay textured side up on a clean flat surface then dose 100 μl (˜2 mg/cm²) of protective composition (not shown) onto the vitro-skin and spread evenly over the surface by rubbing for 30 seconds with a gloved finger.

4. Using a scalpel blade cut the vitro-skin segment (3) into two equal sections, each large enough to completely cover the top of the cell. Place the relevant size o-ring (5) (22 mm, for the specified Franz-cell) onto each section of the vitro skin and dose to 150 mg/cm² of depilatory composition (4) (“Veet Normal Skin Hair Removal Cream” or an equivalent (an equivalent being a composition comprising 3.7% wt thioglycolic acid)) into the centre then, using a glass rod, evenly spread the cream around the inside of the o-ring (5). Using tweezers pick up the vitro-skin segment and place the vitro-skin segment, depilatory and o-ring centrally over the receptor cell (2), place donor cell (1) over the top and clamp in place. Turn on stirrer plate and start 10 minute countdown timer. After 10 minutes; turn off stirrer and remove the clamp, donor cell (1) and vitro-skin segment and place the receptor solution in a suitable container for analysis.

5. A reference sample should also be run without protective composition treatment on the vitro-skin. Remove a sheet of vitro-skin from the hydration chamber and lay textured side up on a clean flat surface. Repeat step 4 of the protocol to produce the reference sample.

### Sample Analysis

For RP-HPLC analysis, prepare a 50 mM Formic acid (98%+ Fluka) solution and mix 970 ml of this solution with 30 ml acetonitrile (HPLC grade) to act as a mobile phase during the analysis.

A reference standard solution should be made with a concentration of Calcium Thioglycolate Trihydrate of 0.94 mg/ml.

Install a Waters Atlantis T3 3 μm 4.6×50 mm column into the HPLC (although any silica-based C₁₈ reversed phase RP-HPLC column may be used), and ensure all solvent lines for the RP-HPLC are primed and free of leaks. Allow the mobile phase to circulate through the system for 25 minutes at 0.7 mL/Min in order to equilibrate the column. Detection of the thioglycolic acid is via UV spectroscopy.

The RP-HPLC conditions are as follows:
Injection volume: 20 μL
Mobile phase flow rate: 0.70 ml/min
Run time: 10 minutes
UV Detection wavelength: 240 nm
Column temperature: 35° C.
UV sampling rate: ≧5 per second
Retention time: Thioglycolic Acid ˜2.5 min

### Calculations:

Calculate the concentration of Thioglycolic Acid in the sample

$$concentration ( mg \text{/} ml ) = \frac{weight of std ( mg ) \times purity}{25} \times \frac{3}{25}$$

Calculate the concentration of thioglycolic acid in the sample using the following formula:

concentration(mg/ml)=A/B×C×E/F

### Where,

### A=Peak Area of Thioglycolic Acid Sample

### B=Average Peak Area of Thioglycolic Acid Standard

C=Thioglycolic Acid final STD concentration in mg/ml (0.94 mg/ml)
E=Molecular weight of Thioglycolic acid (92.12 g/mol)
F=Molecular weight of Calcium thioglycolate (184.23 g/mol)

The efficacy of the barrier provided by the protective composition (resistance to TGA penetration) can be calculated as a percentage decrease in TGA in the receptor solution:

$$\% reduction = \frac{concentration without {barrier}^{*} - concentration with {barrier}^{*}}{concentration without {barrier}^{*}} \times 100$$

*protective composition

For example, if TGA in solution without protective composition=75 μg/ml and TGA in solution with barrier=15 μg/ml

$$\% reduction = \frac{75 - 60}{75} \times 100 = 85 \%$$

A reduction of TGA penetration of 45% or more is believed to correlate to a significant and user-noticeable reduction in irritation.

### Examples

| | | % Reduction in Thioglycolic |
|---|---|---|
| | | Acid Penetration According to |
| Example | Protective Composition | the Franz Cell Method |
| Example 1 | 100% petrolatum with cone | 61.0 |
| | penetration of 170 | |
| Comparative | 100% Petrolatum with cone | 41.0 |
| Example 1 | penetration of 240 | |
| Comparative | 100% Mineral oil | 25.0 |
| Example 2 | | |
| Comparative | 100% Sunflower Seed oil | 10.8 |
| Example 3 | | |
| Comparative | 100% Olive oil | 0.0 |
| Example 4 | | |

As demonstrated by the Franz Cell data, oils, on their own (see Comparative Examples 2-4) provide little to no barrier to thioglycolic acid and even petrolatum with a cone penetration outside the defined range provides a relatively poor barrier, whereas a petrolatum having a cone penetration in the defined range gives a dramatically superior barrier to penetration (see the Example).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of removing hair from skin, comprising the steps of:
(a) applying a protective composition to an area of skin on which unwanted hair is growing, wherein the protective composition comprises at least 75% petrolatum by weight of the protective composition, the protective composition having a cone penetration of 160 to 210;
(b) applying a depilatory composition to the area of skin to which the protective composition has been applied, the depilatory composition comprising a keratin reducing agent.

2. The method of claim 1, wherein the protective composition comprises at least 80% petrolatum by weight of the protective composition.

3. The method of claim 1, wherein the protective composition comprises at least 95% petrolatum by weight of the protective composition.

4. The method of claim 1, wherein the protective composition comprises from 1% to 25% of powder by weight of the protective composition.

5. The method of claim 1, wherein the protective composition comprises from 5% to 10% of powder by weight of the protective composition.

6. The method of claim 1, wherein the particles comprised within the powder have an average particle size from 0.05 μm to 200 μm.

7. The method of claim 1, wherein the particles comprised within the powder have an average particle size from 0.2 μm to 60 μm.

8. The method of claim 1, comprising the following additional step between step (a) and step (b):
(a1) massaging the protective composition into the skin for at least 10 seconds.

9. The method of any preceding claim, comprising the following additional step immediately following step (b):
(c) leaving the depilatory composition in place on the protective composition for a period of at least 1 minute.

10. The method of claim 1, wherein the keratin reducing agent is selected from the group consisting of potassium thioglycolate, calcium thioglycolate and mixtures thereof.

11. A depilatory kit comprising:
(a) a protective composition, the protective composition comprising at least 75% petrolatum by weight of the protective composition, the protective composition having a cone penetration of 160 to 210;
(b) a depilatory composition comprising an effective amount of a keratin reducing agent.

12. The kit of claim 10, wherein the protective composition comprises at least 80%, petrolatum by weight of the protective composition.

13. The kit of claim 10, wherein the protective composition comprises at least 95% petrolatum by weight of the protective composition.

14. The depilatory kit of claim 10, wherein the protective composition comprises from 1% to 25% of powder by weight of the protective composition.

15. The depilatory kit of claim 10, wherein the protective composition comprises from 5% to 10% of powder by weight of the protective composition.

16. The depilatory kit of claim 10, wherein the powder has an average particle size from 0.5 μm to 200 μm, preferably from 1 μm to 130 μm, more preferably from 5 μm to 30 μm.

17. The depilatory kit of claim 10, wherein the powder has an average particle size from 5 μm to 30 μm.

18. The depilatory kit of claim 10, additionally comprising a tool, such as a scraper or a spatula, or a wipe.

19. The depilatory kit of claim 10, additionally comprising the following instructions:
(a) An instruction to apply the protective composition over an area of skin to be depilated, preferably an instruction to apply the protective composition over an area which is greater than the area of skin to be depilated;
(b) Optionally an instruction to massage the protective composition into the area of skin to be depilated for at least 10 seconds;
(c) An instruction to apply the depilatory composition to the pre-treated area of skin to be depilated;
(d) Optionally, an instruction to leave the depilatory composition in place on top of the protective composition for a period of at least 1 minute;
(e) Optionally an instruction to remove both the protective composition and the depilatory composition from the skin, by scraping, wiping or rubbing it off.
(f) Optionally an instruction to treat the area of skin which has been depilated with a post-treatment skin care composition.
